(19) United States
(12) Patent Application Publication
(10) Pub. No.: US 2025/0186156 A1
(43) Pub. Date: Jun. 12, 2025

(54) DETECTION DEVICE

(71) Applicant: HTC Corporation, Taoyuan City (TW)

(72) Inventors: Chun-Yih WU, Taoyuan City (TW); Ta-Chun PU, Taoyuan City (TW); Yen-Liang KUO, Taoyuan City (TW)

(73) Assignee: HTC Corporation, Taoyuan City (TW)

(21) Appl. No.: 18/409,453

(22) Filed: Jan. 10, 2024

(30) Foreign Application Priority Data
Dec. 6, 2023 (TW) 112147479

(51) Int. Cl. A61B 90/00; A61B 5/00; A61B 5/01; H04B 13/00; H04L 5/00

(52) U.S. Cl. CPC A61B 90/06; A61B 5/0028; A61B 5/01; H04B 13/005; H04L 5/0048; A61B 2562/029

(57) ABSTRACT

A detection device includes an HBC (Human Body Communication) module, a first coupler, a first ADC (Analog-to-Digital Converter), an electrode element, a second coupler, a second ADC, a signal processor, and a tunable matching circuit. The HBC module generates an HBC signal. The first coupler generates a first branch signal according to the HBC signal. The first ADC converts the first branch signal into a first digital signal. The electrode element receives a physiological signal from a human body portion. The second coupler generates a second branch signal according to the physiological signal. The second ADC converts the second branch signal into a second digital signal. The signal processor generates a control signal according to the first digital signal and the second digital signal, thereby adjusting the impedance value of the tunable matching circuit.

### CROSS REFERENCE TO RELATED APPLICATIONS

This application claims priority of Taiwan Patent Application No. 112147479 filed on Dec. 6, 2023, the entirety of which is incorporated by reference herein.

### BACKGROUND OF THE INVENTION

### Field of the Invention

The invention relates to a detection device, and more particularly, to a detection device for detecting a human portion.

### Description of the Related Art

Since the conductivity of the human body is affected by many factors (e.g., skin condition, humidity, temperature, etc.), overall detection accuracy in the field of biological detection may be seriously degraded. Accordingly, there is a need to propose a novel solution for solving this problem of the prior art.

### BRIEF SUMMARY OF THE INVENTION

In an exemplary embodiment, the invention is directed to a detection device for detecting a human body portion. The detection device includes an HBC (Human Body Communication) module, a first coupler, a first ADC (Analog-to-Digital Converter), an electrode element, a second coupler, a second ADC, a signal processor, and a tunable matching circuit. The HBC module generates an HBC signal. The first coupler generates a first branch signal according to the HBC signal. The first ADC converts the first branch signal into a first digital signal. The electrode element receives a physiological signal from a human body portion. The second coupler generates a second branch signal according to the physiological signal. The second ADC converts the second branch signal into a second digital signal. The signal processor generates a control signal according to the first digital signal and the second digital signal. The tunable matching circuit is coupled between the first coupler and the second coupler. The impedance value of the tunable matching circuit is selectively adjusted according to the control signal.

In some embodiments, the human body portion is human skin.

In some embodiments, the electrode element directly touches the human body portion.

In some embodiments, each of the first coupler and the second coupler is a directional coupler or a power splitter.

In some embodiments, if the strength ratio of the second digital signal to the first digital signal is smaller than or equal to a threshold value, the impedance value of the tunable matching circuit will be kept unchanged.

In some embodiments, if the strength ratio of the second digital signal to the first digital signal is greater than the threshold value, the impedance value of the tunable matching circuit will be changed according to the control signal.

In some embodiments, the threshold value is about 5% or 10%.

In some embodiments, the detection device further includes a sensor. The sensor detects the physiological information of the human body portion, so as to generate a detection signal.

In some embodiments, the physiological information includes a temperature data and/or a humidity data.

In some embodiments, the signal processor generates the control signal further in view of the detection signal.

In another exemplary embodiment, the invention is directed to a detection device for detecting a human body portion. The detection device includes an HBC module, a transmitter, a receiver, a signal processor, and a tunable matching circuit. The HBC module generates an HBC signal. The transmitter transmits a reference carrier-wave signal to the human body portion according to the HBC signal. The receiver receives a reflection carrier-wave signal from the human body portion. The signal processor generates a control signal according to the reference carrier-wave signal and the reflection carrier-wave signal. The tunable matching circuit is coupled between the transmitter and the receiver. The impedance value of the tunable matching circuit is selectively adjusted according to the control signal.

In some embodiments, each of the reference carrier-wave signal and the reflection carrier-wave signal is a wireless signal.

In some embodiments, the operational frequency of each of the reference carrier-wave signal and the reflection carrier-wave signal is from 1 MHz to 200 MHz.

In some embodiments, the signal processor further compares the reflection carrier-wave signal with the reference carrier-wave signal, so as to obtain a differential data.

In some embodiments, the differential data includes an amplitude difference between the reflection carrier-wave signal and the reference carrier-wave signal.

In some embodiments, the differential data further includes a phase difference between the reflection carrier-wave signal and the reference carrier-wave signal.

In some embodiments, the differential data includes an RSSI (Received Signal Strength Indication) difference between the reflection carrier-wave signal and the reference carrier-wave signal.

In some embodiments, the differential data further includes a PDR (Packet Delivery Rate) difference between the reflection carrier-wave signal and the reference carrier-wave signal.

In some embodiments, if the reflection carrier-wave signal is substantially different from the reference carrier-wave signal, the impedance value of the tunable matching circuit will be kept unchanged.

In some embodiments, if the reflection carrier-wave signal is substantially the same as the reference carrier-wave signal, the impedance value of the tunable matching circuit will be changed according to the control signal.

### BRIEF DESCRIPTION OF DRAWINGS

The invention can be more fully understood by reading the subsequent detailed description and examples with references made to the accompanying drawings, wherein:

FIG. 1 is a diagram of a detection device according to an embodiment of the invention;

FIG. 2 is a diagram of a detection device according to an embodiment of the invention; and

FIG. 3 is a flowchart of a detection device according to another embodiment of the invention.

### DETAILED DESCRIPTION OF THE INVENTION

In order to illustrate the foregoing and other purposes, features and advantages of the invention, the embodiments and figures of the invention will be described in detail as follows.

Certain terms are used throughout the description and following claims to refer to particular components. As one skilled in the art will appreciate, manufacturers may refer to a component by different names. This document does not intend to distinguish between components that differ in name but not function. In the following description and in the claims, the terms “include” and “comprise” are used in an open-ended fashion, and thus should be interpreted to mean “include, but not limited to . . . ”. The term “substantially” means the value is within an acceptable error range. One skilled in the art can solve the technical problem within a predetermined error range and achieve the proposed technical performance. Also, the term “couple” is intended to mean either an indirect or direct electrical connection. Accordingly, if one device is coupled to another device, that connection may be through a direct electrical connection, or through an indirect electrical connection via other devices and connections.

The following disclosure provides many different embodiments, or examples, for implementing different features of the subject matter provided. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

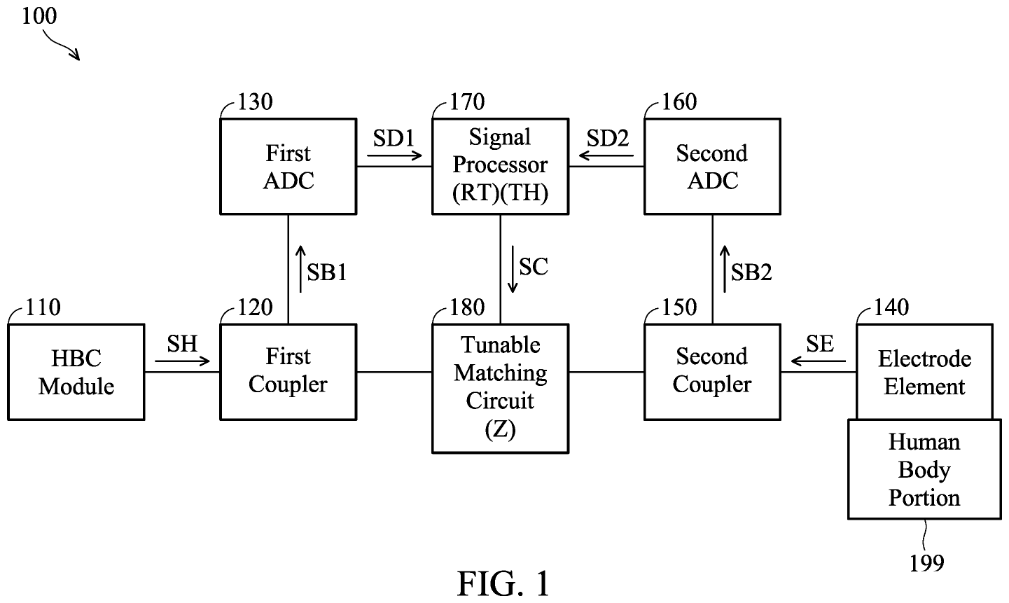

FIG. 1 is a diagram of a detection device 100 according to an embodiment of the invention. The detection device 100 may be applied to a mobile device or a wearable device, such as a smart phone, a tablet computer, a notebook computer, a smart watch, or a pair of AR (Augmented Reality) glasses. In the embodiment of FIG. 1, the detection device 100 includes an HBC (Human Body Communication) module 110, a first coupler 120, a first ADC (Analog-to-Digital Converter) 130, an electrode element 140, a second coupler 150, a second ADC 160, a signal processor 170, and a tunable matching circuit 180. It should be understood that the detection device 100 may further include other components, such as a housing, a speaker, and/or a power supply module, although they are not displayed in FIG. 1.

In some embodiments, the detection device 100 is configured to detect a human body portion 199. The human body portion 199 may be any portion of a user, such as human skin, but it is not limited thereto.

The HBC module 110 generates an HBC signal SH. For example, the operational frequency of the HBC signal SH may be from 1 MHz to 200 MHz. Generally, the HBC signal SH can be transmitted through the first coupler 120, the tunable matching circuit 180, and the second coupler 150 to the electrode element 140. The electrode element 140 may directly touch the human body portion 199. In response, the electrode element 140 receives a physiological signal SE from the human body portion 199. In some embodiments, the physiological signal SE is considered as a reflection signal of the HBC signal SH relative to the human body portion 199.

The first coupler 120 may be a directional coupler or a power splitter. Specifically, the first coupler 120 has a first terminal coupled to the HBC module 110, a second terminal coupled to the first ADC 130, and a third terminal coupled to the tunable matching circuit 180. The first coupler 120 generates a first branch signal SB1 according to the HBC signal SH. The first branch signal SB1 may merely have a small percentage of energy of the HBC signal SH, such as 1%, 5% or 10%. Next, the first ADC 130 converts the first branch signal SB1 into a first digital signal SD1.

The second coupler 150 may be another directional coupler or another power splitter. Specifically, the second coupler 150 has a first terminal coupled to the electrode element 140, a second terminal coupled to the second ADC 160, and a third terminal coupled to the tunable matching circuit 180. The second coupler 150 generates a second branch signal SB2 according to the physiological signal SE. The second branch signal SB2 may merely have a small percentage of energy of the physiological signal SE, such as 1%, 5% or 10%. Next, the second ADC 160 converts the second branch signal SB2 into a second digital signal SD2.

The signal processor 170 is coupled between the first ADC 130 and the second ADC 160. The signal processor 170 generates a control signal SC according to the first digital signal SD1 and the second digital signal SD2. The tunable matching circuit 180 is coupled between the first coupler 120 and the second coupler 150. The impedance value Z of the tunable matching circuit 180 is selectively adjusted according to the control signal SC.

In some embodiments, the signal processor 170 calculates the strength ratio RT of the second digital signal SD2 to the first digital signal SD1, and then compares the strength ratio RT with a threshold value TH. The threshold value TH may be 5% or 10%, but it is not limited thereto. For example, the strength ratio RT may be calculated according to the following equation (1):

$$RT = \frac{SD 2}{SD 1} \qquad ( 1 )$$

where “RT” represents the strength ratio RT, “SD1” represents the signal strength of the first digital signal SD1, and “SD2” represents the signal strength of the second digital signal SD2.

For example, if the strength ratio RT of the second digital signal SD2 to the first digital signal SD1 is smaller than or equal to the threshold value TH, it may imply an ideal case and represent that the physiological signal SE from the human body portion 199 is relatively weak. At the time, the conductivity of the human body portion 199 may be relatively high, and the impedance value Z of the tunable matching circuit 180 can be kept unchanged.

Conversely, if the strength ratio RT of the second digital signal SD2 to the first digital signal SD1 is greater the threshold value TH, it may imply a non-ideal case and represent that the physiological signal SE from the human body portion 199 is relatively strong. At the time, the conductivity of the human body portion 199 may be relatively low, and the impedance value Z of the tunable matching circuit 180 can be changed according to the control signal SC.

With the design of the invention, the proposed detection device 100 touching the human body portion 199 can appropriately adjust its impedance matching according to different states of the human body portion 199, thereby significantly improving the overall detection accuracy.

The following embodiments will introduce different configurations and detail structural features of the detection device 100. It should be understood that these figures and descriptions are merely exemplary, rather than limitations of the invention.

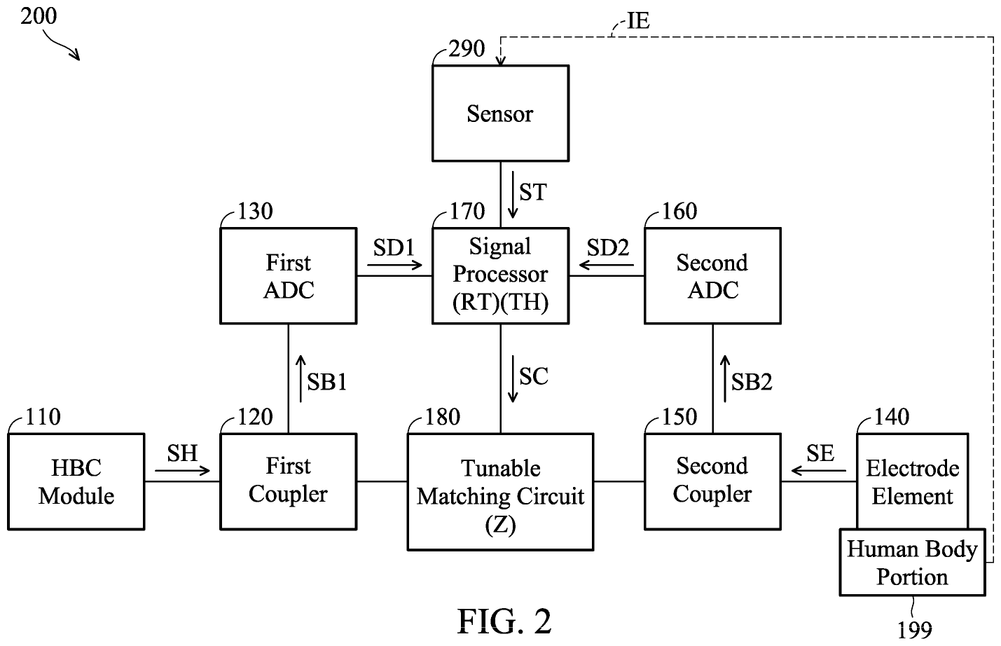

FIG. 2 is a diagram of a detection device 200 according to an embodiment of the invention. FIG. 2 is similar to FIG. 1. In the embodiment of FIG. 2, the detection device 200 further includes a sensor 290 coupled to the signal processor 170. The sensor 290 detects the physiological information IE of the human body portion 199, so as to generate a detection signal ST. For example, the physiological information IE may include a temperature data and/or a humidity data of the human body portion 199, but it is not limited thereto. The physiological information IE may be used as an auxiliary condition for determination. Thus, the signal processor 170 can generate the control signal SC further in view of the detection signal ST. Other features of the detection device 200 of FIG. 2 are similar to those of the detection device 100 of FIG. 1. Accordingly, the two embodiments can achieve similar levels of performance.

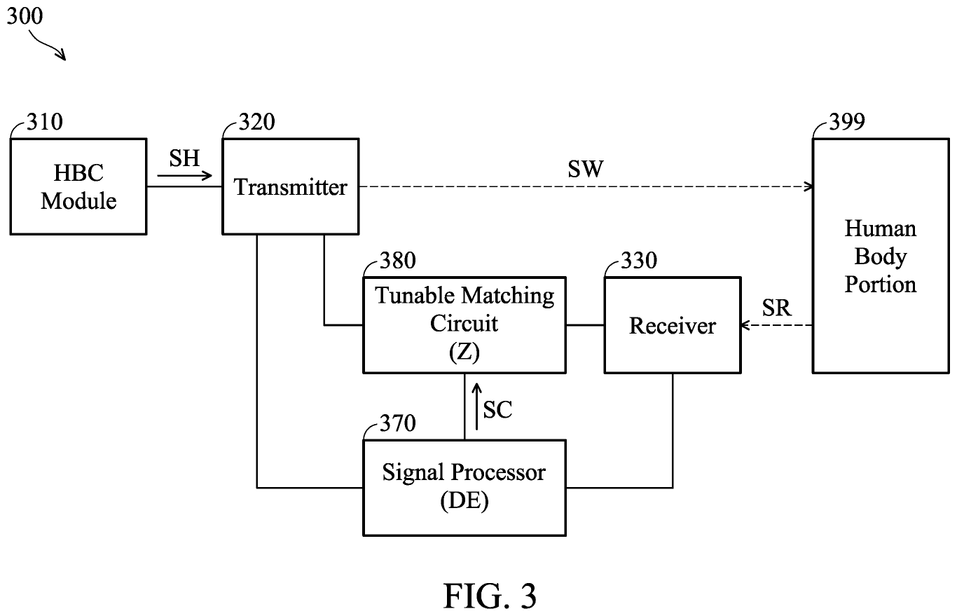

FIG. 3 is a diagram of a detection device 300 according to another embodiment of the invention. The detection device 300 may be applied to a mobile device or a wearable device. In the embodiment of FIG. 3, the detection device 300 includes an HBC module 310, a transmitter (TX) 320, a receiver (RX) 330, a signal processor 370, and a tunable matching circuit 380. It should be understood that the detection device 300 may further include other components although they are not displayed in FIG. 3.

In some embodiments, the detection device 300 is configured to detect a human body portion 399. The human body portion 399 may be any portion of a user, such as human skin, but it is not limited thereto.

The HBC module 310 generates an HBC signal SH. For example, the operational frequency of the HBC signal SH may be from 1 MHz to 200 MHz. The transmitter 320 is coupled to the HBC module 310. The transmitter 320 transmits a reference carrier-wave signal SW to the human body portion 399 according to the HBC signal SH. In response, the receiver 330 receives a reflection carrier-wave signal SR from the human body portion 399. In some embodiments, each of the reference carrier-wave signal SW and the reflection carrier-wave signal SR is a wireless signal. For example, the operational frequency of each of the reference carrier-wave signal SW and the reflection carrier-wave signal SR may be from 1 MHz to 200 MHz.

The signal processor 370 is coupled between the transmitter 320 and the receiver 330. The signal processor 370 generates a control signal SC according to the reference carrier-wave signal SW and the reflection carrier-wave signal SR. The tunable matching circuit 380 is also coupled between the transmitter 320 and the receiver 330. The impedance value Z of the tunable matching circuit 380 is selectively adjusted according to the control signal SC.

In some embodiments, the signal processor 370 further compares the reflection carrier-wave signal SR with the reference carrier-wave signal SW, so as to obtain a differential data DE. Specifically, the differential data DE may be classified as an analog data or a digital data.

In some embodiments, if the differential data DE is classified as the analog data, the differential data DE will include an amplitude difference and/or a phase difference between the reflection carrier-wave signal SR and the reference carrier-wave signal SW. In alternative embodiments, if the differential data DE is classified as the digital data, the differential data DE will include an RSSI (Received Signal Strength Indication) difference and/or a PDR (Packet Delivery Rate) difference between the reflection carrier-wave signal SR and the reference carrier-wave signal SW.

For example, if the signal processor 370 determines that the reflection carrier-wave signal SR is substantially different from the reference carrier-wave signal SW (i.e., the differential data DE is remarkable), it may imply an ideal case and represent that the human body portion 399 absorbs most of the reference carrier-wave signal SW. At the time, the conductivity of the human body portion 399 may be relatively high, and the impedance value Z of the tunable matching circuit 380 can be kept unchanged.

Conversely, if the signal processor 370 determines that the reflection carrier-wave signal SR is substantially the same as the reference carrier-wave signal SW (i.e., the differential data DE is trivial), it may imply a non-ideal case and represent that the human body portion 399 reflects most of the reference carrier-wave signal SW. At the time, the conductivity of the human body portion 399 may be relatively low, and the impedance value Z of the tunable matching circuit 380 can be changed according to the control signal SC.

With the design of the invention, the proposed detection device 300 without touching the human body portion 399 can appropriately adjust its impedance matching according to different states of the human body portion 399, thereby significantly improving the overall detection accuracy.

The invention proposed a novel detection device. In comparison to the conventional design, the invention has at least the advantages of enhancing the overall detection accuracy and reducing the whole manufacturing cost. Therefore, the invention is suitable for application in a variety of devices.

Note that the above element parameters are not limitations of the invention. A designer can fine-tune these setting values according to different requirements. It should be understood that the detection device of the invention is not limited to the configurations of FIGS. 1-3. The invention may include any one or more features of any one or more embodiments of FIGS. 1-3. In other words, not all of the features displayed in the figures should be implemented in the detection device of the invention.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having the same name (but for use of the ordinal term) to distinguish the claim elements.

It will be apparent to those skilled in the art that various modifications and variations can be made in the invention. It is intended that the standard and examples be considered as exemplary only, with a true scope of the disclosed embodiments being indicated by the following claims and their equivalents.

What is claimed is:

1. A detection device for detecting a human body portion, comprising:
an HBC (Human Body Communication) module, generating an HBC signal;
a first coupler, generating a first branch signal according to the HBC signal;
a first ADC (Analog-to-Digital Converter), converting the first branch signal into a first digital signal;
an electrode element, receiving a physiological signal from the human body portion;
a second coupler, generating a second branch signal according to the physiological signal.
a second ADC, converting the second branch signal into a second digital signal;
a signal processor, generating a control signal according to the first digital signal and the second digital signal; and
a tunable matching circuit, coupled between the first coupler and the second coupler, wherein an impedance value of the tunable matching circuit is selectively adjusted according to the control signal.

2. The detection device as claimed in claim 1, wherein the human body portion is human skin.

3. The detection device as claimed in claim 1, wherein the electrode element directly touches the human body portion.

4. The detection device as claimed in claim 1, wherein each of the first coupler and the second coupler is a directional coupler or a power splitter.

5. The detection device as claimed in claim 1, wherein if a strength ratio of the second digital signal to the first digital signal is smaller than or equal to a threshold value, the impedance value of the tunable matching circuit is kept unchanged.

6. The detection device as claimed in claim 5, wherein if the strength ratio of the second digital signal to the first digital signal is greater than the threshold value, the impedance value of the tunable matching circuit is changed according to the control signal.

7. The detection device as claimed in claim 5, wherein the threshold value is about 5% or 10%.

8. The detection device as claimed in claim 1, further comprising:
a sensor, detecting physiological information of the human body portion, so as to generate a detection signal.

9. The detection device as claimed in claim 8, wherein the physiological information comprises a temperature data and/or a humidity data.

10. The detection device as claimed in claim 8, wherein the signal processor generates the control signal further in view of the detection signal.

11. A detection device for detecting a human body portion, comprising:
an HBC (Human Body Communication) module, generating an HBC signal;
a transmitter, transmitting a reference carrier-wave signal to the human body portion according to the HBC signal;
a receiver, receiving a reflection carrier-wave signal from the human body portion;
a signal processor, generating a control signal according to the reference carrier-wave signal and the reflection carrier-wave signal; and
a tunable matching circuit, coupled between the transmitter and the receiver, wherein an impedance value of the tunable matching circuit is selectively adjusted according to the control signal.

12. The detection device as claimed in claim 11, wherein each of the reference carrier-wave signal and the reflection carrier-wave signal is a wireless signal.

13. The detection device as claimed in claim 11, wherein an operational frequency of each of the reference carrier-wave signal and the reflection carrier-wave signal is from 1 MHz to 200 MHz.

14. The detection device as claimed in claim 11, wherein the signal processor further compares the reflection carrier-wave signal with the reference carrier-wave signal, so as to obtain a differential data.

15. The detection device as claimed in claim 14, wherein the differential data comprises an amplitude difference between the reflection carrier-wave signal and the reference carrier-wave signal.

16. The detection device as claimed in claim 15, wherein the differential data further comprises a phase difference between the reflection carrier-wave signal and the reference carrier-wave signal.

17. The detection device as claimed in claim 14, wherein the differential data comprises an RSSI (Received Signal Strength Indication) difference between the reflection carrier-wave signal and the reference carrier-wave signal.

18. The detection device as claimed in claim 17, wherein the differential data further comprises a PDR (Packet Delivery Rate) difference between the reflection carrier-wave signal and the reference carrier-wave signal.

19. The detection device as claimed in claim 11, wherein if the reflection carrier-wave signal is substantially different from the reference carrier-wave signal, the impedance value of the tunable matching circuit is kept unchanged.

20. The detection device as claimed in claim 11, wherein if the reflection carrier-wave signal is substantially the same as the reference carrier-wave signal, the impedance value of the tunable matching circuit is changed according to the control signal.
